(19) United States
(12) Patent Application Publication
(10) Pub. No.: US 2025/0179410 A1
(43) Pub. Date: Jun. 5, 2025

(54) SYSTEM AND METHOD FOR SIMULTANEOUSLY MEASURING REFRACTIVE INDEX AND TURBIDITY

(71) Applicant: Stichting IMEC Nederland, Wageningen (NL)

(72) Inventors: Mark Zentile, Arnhem (NL); Esther Kho, Wageningen (NL); Eva Wentink, Heeze (NL); Vera Verbiest, Rhenen (NL); Xu Zhang, Woudeneberg (NL)

(21) Appl. No.: 18/961,242

(22) Filed: Nov. 26, 2024

(30) Foreign Application Priority Data
Nov. 30, 2023 (EP) 23213193.8

(51) Int. Cl. C12M 1/34; G01N 21/41; G01N 21/51; G01N 33/493

(52) U.S. Cl. CPC C12M 41/30; G01N 21/4133; G01N 21/51; G01N 33/493; G01N 2201/06113; G01N 2201/0846

(57) ABSTRACT

A system for simultaneously measuring refractive index and turbidity of a liquid sample is provided. The system comprises a coherent light source configured to generate a coherent light signal, an optical structure being in contact with the liquid sample, the optical structure is configured to transmit the coherent light signal to the liquid sample and further to receive a light signal from the liquid sample in response to the coherent light signal, and a detector configured to receive the light signal and further to measure refractive index and turbidity of the liquid sample simultaneously from the light signal.

### CROSS-REFERENCE TO RELATED APPLICATIONS

The present application is a non-provisional patent application claiming priority to European Patent Application No. EP 23213193.8, filed Nov. 30, 2023, the contents of which are hereby incorporated by reference.

### FIELD OF THE DISCLOSURE

The present disclosure relates to the simultaneous measurement of refractive index and turbidity of a liquid sample, especially to measure a relationship between dissolved solids and undissolved solids in the liquid sample.

### BACKGROUND

Generally, refractometry may be used to quantify the amount of dissolved solids in a liquid solvent, since the dissolved substances may increase the total refractive index of the solvent. On the other hand, turbidity may be used as a measure of the undissolved solids in the liquid solvent, since the undissolved solids may create a non-homogeneous medium that may cause scattering of incident light.

For example, the document CN 115236034 A discloses a reflective refractometer provided with a prism to measure the refractive index and turbidity of a liquid. However, the refractometry scheme may require a larger active area, for example, to be contacted with the surface of the liquid. Moreover, the document does not utilize the refractive index measurement and turbidity measurement in combination.

### SUMMARY

Accordingly, the present disclosure provides a system and a method for simultaneously measuring refractive index and turbidity of a liquid sample, which can be integrated into smaller spaces. The present disclosure further utilizes the refractive index measurement and turbidity measurement in combination to measure a relationship between dissolved solids and undissolved solids, e.g., the ratio of dissolved to undissolved solids in the liquid sample.

In an example embodiment, a system for simultaneously measuring refractive index and turbidity of a liquid sample is provided. The system comprises a coherent light source configured to generate a coherent light signal. The system further comprises an optical structure being in contact with the liquid sample, the optical structure is configured to transmit the coherent light signal to the liquid sample and further to receive a light signal from the liquid sample in response to the coherent light signal.

Moreover, the system comprises a detector configured to receive the light signal and further to measure refractive index and turbidity of the liquid sample simultaneously from the light signal.

Therefore, the coherent light source and the optical structure for conveying the coherent light may facilitate a much smaller active area, e.g., to be contacted with the surface of the liquid sample to measure the back reflected and/or scattered light in response to the incident coherent light, which can be integrated into smaller spaces.

In an example embodiment, the detector is configured to measure or estimate a concentration of dissolved solids in the liquid sample from the refractive index measurement. The detector may further be configured to measure or estimate a concentration of undissolved solids in the liquid sample from the turbidity measurement.

For example, the measurement of the refractive index may be understood as a measurement of the Fresnel reflection, especially at the surface of the liquid sample. On the other hand, the measurement of turbidity may be understood as the measurement of scattered light directed from the liquid sample in the same optical mode as the Fresnel reflection, where the intensity of the combination of Fresnel reflection and scattered light fluctuates over time, dependent on the turbidity of the sample.

For example, the term estimation can be understood as an approximation of the measurements with respect to reference measurements. For instance, the refractive index and/or turbidity measurements may be correlated or compared with reference refractive index and/or turbidity measurements corresponding to known concentrations of dissolved and/or undissolved solids in a reference liquid sample.

In an example embodiment, the simultaneous measurements of refractive index and turbidity may be utilized in combination, e.g., to measure the ratio of dissolved to undissolved solids in the liquid sample.

In an example embodiment, the concentration of dissolved solids in the liquid sample is a concentration of dissolved metabolites in a urine sample. In addition, the concentration of undissolved solids in the liquid sample is a concentration of secreted proteins, e.g., the fraction of undissolved proteins, in the urine sample. The concentration of undissolved solids may be caused by blood cells, mucus, crystals etc. in the urine sample, thereby increasing the turbidity.

Some example embodiments allow for a simultaneous estimation of protein content and specific gravity (USG) of the urine sample, and their ratio can be used as a possible parameter to yield proteinuria diagnosis.

In an example embodiment, the concentration of dissolved solids in the liquid sample is a concentration of a substrate in a bioreactor. Additionally, the concentration of undissolved solids in the liquid sample is a concentration of cells in suspension in the bioreactor.

Some example embodiment allow for a simultaneous measurement of cell concentration and substrate concentration in the bioreactor, and their ratio can be used to calculate the optimal cell growth rate and/or the nutrient consumption rate in the bioreactor.

In an example embodiment, the concentration of dissolved solids in the liquid sample may be a concentration of dissolved sugars in a fruit juice sample. Further alternatively, the concentration of dissolved solids in the liquid sample may be a concentration of dissolved salts in a water sample.

In an example embodiment, the light signal comprises one or more lights reflected at a surface of the liquid sample and/or one or more lights scattered within the liquid sample, especially in response to the coherent light signal. For instance, the back reflected and/or scattered lights may be in the same spatial mode of the coherent light signal.

In an example embodiment, the optical structure comprises a single optical fiber configured to transmit the coherent light signal to the liquid sample and further to receive the light signal from the liquid sample. In this regard, the single optical fiber is a single mode optical fiber.

In an example embodiment, the single optical fiber is configured such that an end of the single optical fiber is immersed in the liquid sample.

For example, a much smaller active area can be achieved by using only one single mode optical fiber, which may have a core diameter of 8 μm to 10 μm.

In an example embodiment, the optical structure comprises an optical circulator configured to direct the coherent light signal towards the liquid sample and further to direct the light signal from the liquid sample.

For example, the optical circulator may be a one-directional single mode fiber optic circulator, which may provide for bidirectional propagation of light in the single optical fiber.

In an example embodiment, the system further comprises a signal generator configured to modulate the coherent light source, which may be at a predefined frequency. In various examples, a set of parameters, such as angle, power, phase and polarization, of the coherent light can be controlled.

In an example embodiment, the system comprises an optical switch, and a plurality of optical structures. In this regard, the optical switch is configured such that each of the plurality of optical structures transmits a respective coherent light signal from the coherent light source to the liquid sample and further to receive a respective light signal from the liquid sample in response to the coherent light signal.

Moreover, the detector is configured to receive the light signals from the plurality of optical structures and further to measure refractive index and turbidity of the liquid sample simultaneously from each of the light signals.

In an example embodiment, for example, a plurality of single mode fibers can be multiplexed via the optical switch without needing additional light sources and detectors, which may result in a cost-effective scheme.

For example, the plurality of single mode fibers can be used for a single liquid sample, e.g., to achieve multiple simultaneous measurements of the refractive index and turbidity at different surface locations of the liquid sample (e.g., for measuring a difference of the measurements at different locations) or at different timing instances (e.g., for measuring a change over time).

In an example embodiment, the plurality of single mode fibers can be used for a corresponding plurality of liquid samples to simultaneously measure refractive index and turbidity of the respective liquid samples.

In an example embodiment, the detector is configured to measure a resultant reflection from the light signal corresponding to an interference between lights due to Fresnel reflection and lights from scattering in the liquid sample, especially due to the transmission of the coherent light signal to the liquid sample.

In this regard, the detector is configured to measure the Fresnel reflection by averaging the measured resultant reflection in order to measure the refractive index of the liquid sample. The detector may be configured to measure the scattering by filtering or quantifying or digitally processing the measured resultant reflection in order to measure the turbidity of the liquid sample.

For example, the detector may comprise a photodetector to detect the light signal received from the optical structure. For example, the photodetector may be a silicon, In GaAs, or GaAs based laser power photodetector, which may facilitate a fast response and less sensitivity to the temperature.

For example, the coherent light source may be a laser light source, e.g., a laser diode.

In another example embodiment, a method for simultaneously measuring refractive index and turbidity of a liquid sample is provided. The method comprises the steps of generating a coherent light signal, transmitting the coherent light signal to the liquid sample, receiving a light signal from the liquid sample in response to the coherent light signal, and measuring refractive index and turbidity of the liquid sample simultaneously from the light signal.

In an example embodiment, the method further comprises the step of measuring or estimating a concentration of dissolved solids in the liquid sample from the refractive index measurement. The method may further comprises the step of measuring or estimating a concentration of undissolved solids in the liquid sample from the turbidity measurement.

It is to be noted that the method according to the second aspect corresponds to the system according to the first aspect and its implementation forms. Accordingly, the method of the second aspect may have corresponding implementation forms. Further, the method of the second aspect achieves the same advantages and effects as the system of the first aspect and its respective implementation forms.

### BRIEF DESCRIPTION OF THE DRAWINGS

Exemplary embodiments of the present disclosure are now further explained with respect to the drawings by way of example only, and not for limitation. In the drawings:

FIG. 1 shows an exemplary embodiment of the system according to the present disclosure;

FIG. 2 shows an exemplary embodiment of the detector;

FIG. 3 shows another exemplary embodiment of the system according to the present disclosure;

FIG. 4 shows an exemplary embodiment of the system according to the present disclosure;

FIG. 5 shows an exemplary embodiment of the system according to the present disclosure;

FIG. 6 shows an exemplary embodiment of the system according to the present disclosure;

FIG. 7 shows an exemplary electric field distribution in the liquid sample due to reflection and scattering;

FIG. 8 shows exemplary photodetector signals from two Formazin solutions;

FIG. 9 shows exemplary photodetector signals from two Formazin solutions for modulated light sources;

FIG. 10 shows an exemplary graph of turbidity vs USG for human urine samples; and

FIG. 11 shows an exemplary embodiment of the method according to the present disclosure.

### DETAILED DESCRIPTION

Reference will now be made in detail to the embodiments of the present disclosure, examples of which are illustrated in the accompanying drawings. However, the following embodiments of the present disclosure may be variously modified and the range of the present disclosure is not limited by the following embodiments.

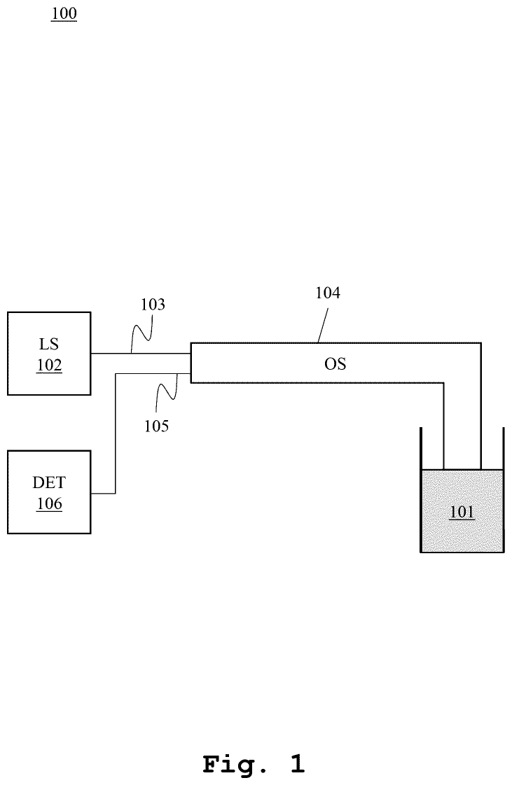

In FIG. 1, as exemplary embodiment of the system 100 according to the present disclose is illustrated. The system 100 may simultaneously measure refractive index and turbidity of a liquid sample 101, i.e., may simultaneously act as a refractometer and a turbidimeter.

In this regard, the system 100 may comprise a coherent light source 102 that may generate a coherent light signal 103. For example, the coherent light source 102 may be a laser light source, e.g., a laser diode, which may generate laser beams at a wavelength of 1550 nm.

In addition, the system 100 may comprise an optical structure 104 operably coupled, e.g., optically coupled, to the coherent light source 102 in order to receive and to transmit the coherent light signal 103 through the length of the optical structure 104.

The optical structure 104 may be further in contact with the liquid sample 101, e.g., an end of the optical structure 104 opposite to the end receiving the coherent light signal 103 may be optically coupled with or be immersed in the liquid sample 101.

As such, the optical structure 104 may transmit the coherent light signal 103 to the liquid sample 101 and may receive a return light signal 105, e.g., a back reflected and/or scattered light signal, from the liquid sample 101 in response to the coherent light signal 103.

For example, the return light signal 105 may comprise one or more lights reflected at a surface of the liquid sample 101 in response to the coherent light signal 103 and/or one or more lights scattered within the liquid sample 101 in response to the coherent light signal 103. In this regard, the back reflected and/or scattered lights may be in the same spatial mode of the coherent light signal 103.

The system 100 may further comprise a detector 106 operably coupled, e.g., optically coupled, to the optical structure 104. In this regard, the optical structure 104 may transmit the return light signal 105 from the liquid sample 101 to the detector 106, and the detector may receive and/or detect the return light signal 105 from the optical structure 104.

For instance, the detector 106 may simultaneously measure refractive index and turbidity of the liquid sample 101 from the return light signal 105. In other words, the detector 106 may simultaneously perform a refractive index measurement and a turbidity measurement of the liquid sample 101 from the return light signal 105.

For example, the detector 106 may measure or estimate a concentration of dissolved solids in the liquid sample 101, especially from the refractive index measurement. In addition, the detector 106 may measure or estimate a concentration of undissolved solids in the liquid sample 101 from the turbidity measurement.

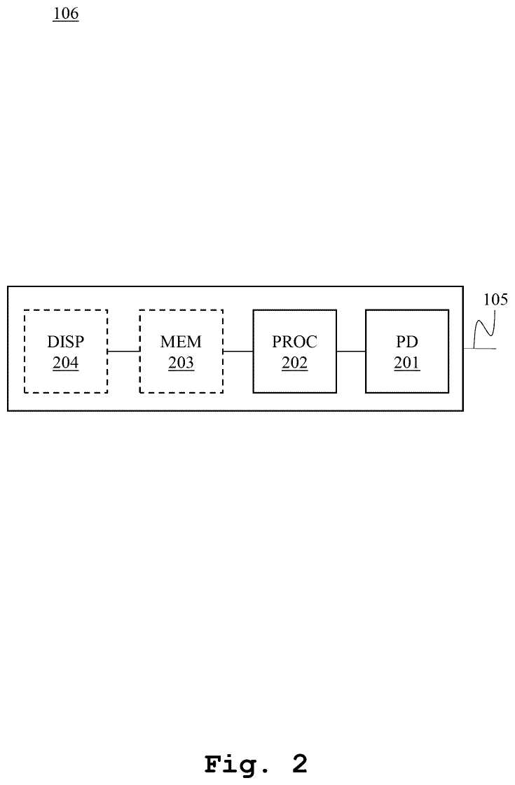

In FIG. 2, an exemplary embodiment of the detector 106 is illustrated. The detector 106 may comprise a photodetector 201, e.g., a photodiode, which may detect the return light signal 105 from the optical structure 104. The detector 106 may further comprise a processor 202 operably coupled to the photodetector 201 and may receive photodetector signals, e.g., photodetector voltage, therefrom.

The processor 202 may process the photodetector signals to perform the refractive index and turbidity measurements of the liquid sample 101, and may correlate the measurements to estimate the concentrations of dissolved and undissolved solids in the liquid sample 101.

For example, the processor 202 may comprise one or more application specific integrated circuits (ASICs), digital signal processors (DSPs), digital signal processing devices (DSPDs), programmable logic devices (PLDs), field programmable gate arrays (FPGAs), controllers, microcontrollers, microprocessors, or the like.

The detector 106 may optionally comprise a memory 203 operably coupled to the processor 202 and/or to the photodetector 201. The memory 203 may store the measurement data from the photodetector 201 and/or the processed measurement data from the processor 202 and/or user instructions for the processor 202.

For example, the memory 203 may comprise random access memory (RAM) units such as static RAM (SRAM) units and dynamic RAM (DRAM) units, register file units, content addressable memory (CAM) units, read only memory (ROM) units, programmable read only memory (PROM) units, removable memory units such as flash memory devices, or the like.

The detector 106 may optionally comprise a display unit or display 204 operably coupled to the memory 203 and/or to the processor 202. The display 204 may allow a user to view the stored measurement data in the memory 203 and/or the processed measurement data from the processor 202, especially in the form of text and/or graphics.

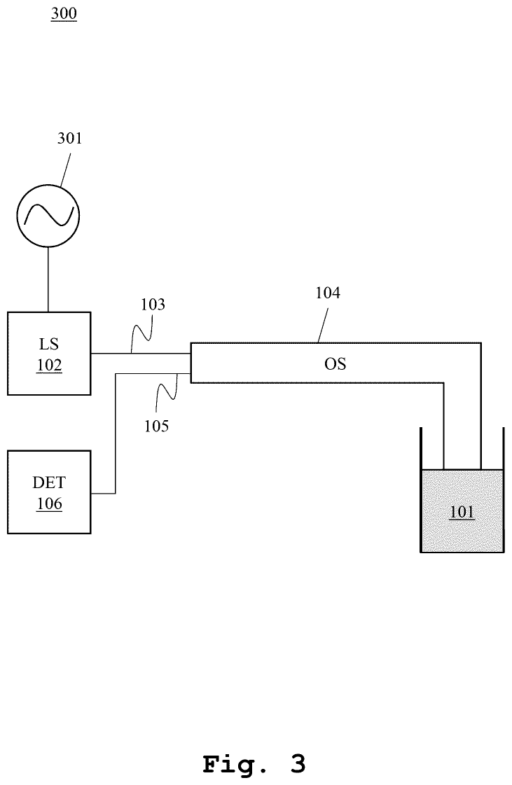

In FIG. 3, an exemplary embodiment of the system 300 according to the present disclosure is illustrated. The system 300 differs from the system 100 in that the system 300 may additionally comprise a signal generator 301 operably coupled to the coherent light source 102. For example, the signal generator 301 may comprise or be a sine wave generator that may modulate the coherent light source 102 at a predefined frequency, especially to generate a modulated coherent light signal.

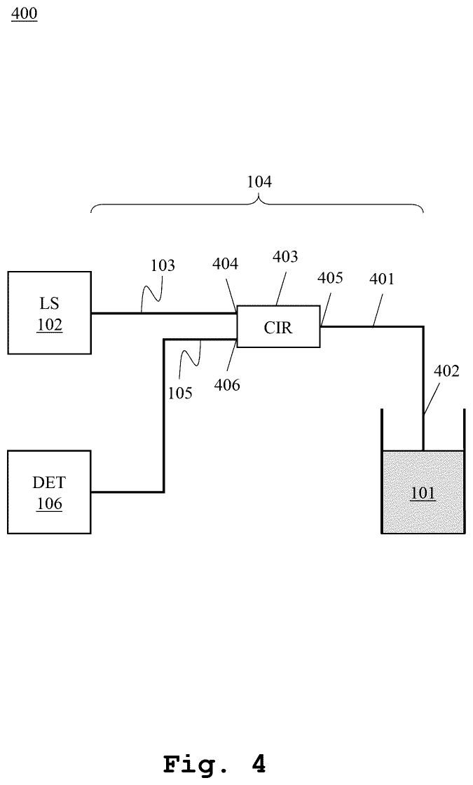

In FIG. 4, an exemplary embodiment of the system 400 according to the present disclosure is illustrated. The system 400 differs from the system 100 in that the optical structure 104 of the system 400 may comprise a single optical fiber 401 and an optical circulator 403. For example, the optical circulator 403 may be operably coupled, e.g., optically coupled, to the coherent light source 102 and to the detector 106. In addition, an end 402 of the single optical fiber 401 may be immersed in the liquid sample 101.

For instance, the optical circulator 403 may be a three-port optical circulator, especially comprising a first port 404, a second port 405 and a third port 406. In this regard, the optical circulator 403 may be arranged with the single optical fiber 401 such that the coherent light signal 103 may be transmitted from the first port 404 to the second port 405 and further to the liquid sample 101, especially through the single optical fiber 401.

Accordingly, the return light signal 105 may be back transmitted from the second port 405 to the third port 406 and further to the detector 106, especially through the single optical fiber 401. In this regard, the optical circulator 403 may be a single mode optical circulator and/or the single optical fiber 401 may be a single mode optical fiber.

Although not shown, the system 400 may additionally comprise the signal generator 301 of FIG. 3 in order to modulate the coherent light source 102.

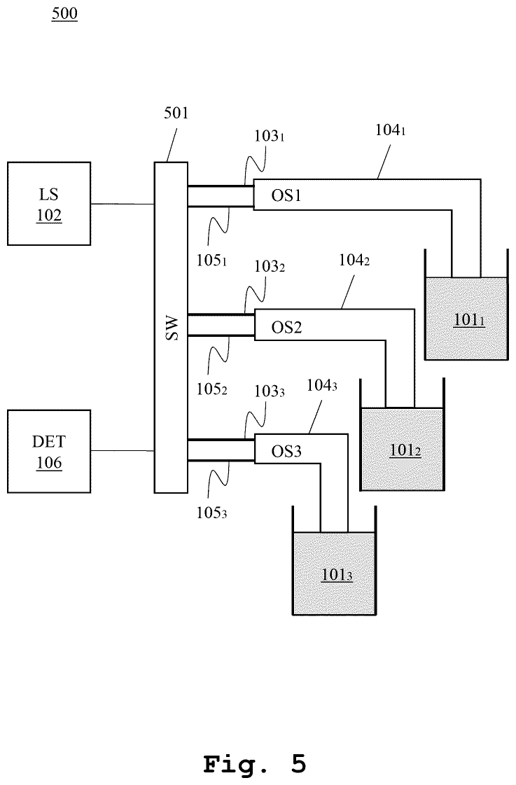

In FIG. 5, another exemplary embodiment of the system 500 according to the present disclosure is illustrated. The system 500 differs from the system 100 in that the system 500 may comprise an optical switch 501 and a plurality of optical structures, exemplarily a first optical structure 1041, a second optical structure 1042, and a third optical structure 1043. It should be understood that the number of optical structures can be more than or less than three, as exemplified herein.

For example, the optical switch 501 may be operably coupled, e.g., optically coupled, to the coherent light source 102 and further to the detector 106. In this regard, the optical switch 501 may receive a first coherent light signal 1031, a second coherent light signal 1032, and a third coherent light signal 1033 from the coherent light source 102, especially in a time-multiplexed manner.

For example, the first coherent light signal 1031, the second coherent light signal 1032, and the third coherent light signal 1033 may be identical signals and may correspond to the coherent light signal 103 of the systems 100, 300, and 400. Alternatively, the first coherent light signal 1031, the second coherent light signal 1032, and the third coherent light signal 1033 may be different signals, e.g., in terms of power and/or phase.

For instance, the first optical structure 1041 may be operably coupled, e.g., optically coupled, to the optical switch 501 in order to receive and to transmit the first coherent light signal 1031 through the length of the first optical structure 1041. The first optical structure 1041 may be further in contact with a first liquid sample 1011, e.g., an end of the first optical structure 1041 opposite to the end receiving the first coherent light signal 1031 may be optically coupled with or be immersed in the first liquid sample 1011.

As such, the first optical structure 1041 may transmit the first coherent light signal 1031 to the first liquid sample 1011 and may receive a first return light signal 1051, e.g., a back reflected and/or scattered light signal, from the first liquid sample 1011 in response to the first coherent light signal 1031. The back reflected and/or scattered lights may be in the same spatial mode of the first coherent light signal 1031.

Similarly, the second optical structure 1042 may be operably coupled, e.g., optically coupled, to the optical switch 501 in order to receive and to transmit the second coherent light signal 1032 through the length of the second optical structure 1042. The second optical structure 1042 may be further in contact with a second liquid sample 1012, e.g., an end of the second optical structure 1042 opposite to the end receiving the second coherent light signal 1032 may be optically coupled with or be immersed in the second liquid sample 1012.

As such, the third optical structure 1043 may transmit the third coherent light signal 1033 to the second liquid sample 1012 and may receive a second return light signal 1052, e.g., a back reflected and/or scattered light signal, from the second liquid sample 1012 in response to the second coherent light signal 1032. The back reflected and/or scattered lights may be in the same spatial mode of the second coherent light signal 1032.

Similarly, the third optical structure 1043 may be operably coupled, e.g., optically coupled, to the optical switch 501 in order to receive and to transmit the third coherent light signal 1033 through the length of the third optical structure 1043. The third optical structure 1043 may be further in contact with a third liquid sample 1013, e.g., an end of the third optical structure 1043 opposite to the end receiving the third coherent light signal 1033 may be optically coupled with or be immersed in the third liquid sample 1013.

As such, the third optical structure 1043 may transmit the third coherent light signal 1033 to the third liquid sample 1013 and may receive a third return light signal 1053, e.g., a back reflected and/or scattered light signal, from the third liquid sample 1013 in response to the third coherent light signal 1033. The back reflected and/or scattered lights may be in the same spatial mode of the third coherent light signal 1033.

For example, the optical switch 501 may transmit the first return light signal 1051, the second return light signal 1052, and the third return light signal 1053 to the detector 106, especially in the time-multiplexed manner. In other words, the detector 106 may receive and/or detect the first return light signal 1051, the second return light signal 1052, and the third return light signal 1053 in the time-multiplexed manner via the optical switch 501.

Accordingly, the detector 106 may simultaneously measure refractive index and turbidity of the first liquid sample 1011 from the first return light signal 1051, may simultaneously measure refractive index and turbidity of the second liquid sample 1012 from the second return light signal 1052, and may simultaneously measure refractive index and turbidity of the third liquid sample 1013 from the third return light signal 1053.

Although not shown, the system 500 may additionally comprise the signal generator 301 of FIG. 3 in order to modulate the coherent light source 102. In this regard, the first coherent light signal 1031, the second coherent light signal 1032, and the third coherent light signal 1033 may be modulated at different frequencies.

Although not shown, each of the first optical structure 1041, the second optical structure 1042, and the third optical structure 1043 may comprise a single optical fiber 401 and an optical circulator 403, e.g., as shown in FIG. 4 for the system 400.

Although not shown, each the first optical structure 1041, the second optical structure 1042, and the third optical structure 1043 may be in contact with a common liquid sample 101, e.g., as shown in FIG. 1 for the system 100. In this regard, the end of the first optical structure 1041, the end of the second optical structure 1042, and the end of the third optical structure 1043 may be immersed in the liquid sample 101 at different locations, e.g., spatial surface locations.

In this regard, at a first time instant, the detector 106 may simultaneously measure refractive index and turbidity of the liquid sample 101 from the first return light signal 1051. Furthermore, at a second time instant, the detector 106 may simultaneously measure refractive index and turbidity of the liquid sample 101 from the second return light signal 1052. Moreover, at a third time instant, the detector 106 may simultaneously measure refractive index and turbidity of the liquid sample 101 from the third return light signal 1053.

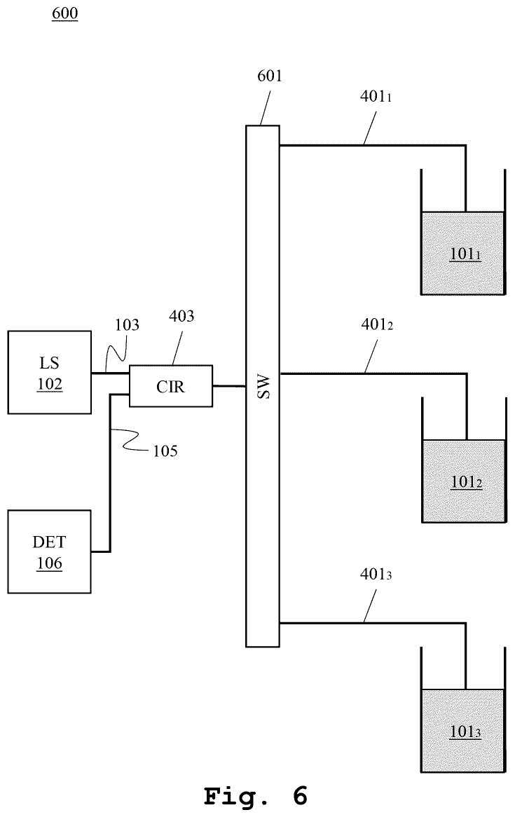

In FIG. 6, an exemplary embodiment of the system 600 according to the present disclosure is illustrated. The system 600 differs from the system 500 in that the system 600 may comprise a single optical circulator 403 optically coupled to an optical switch 601, which is further optically coupled to a plurality of optical fibers, especially single mode optical fibers, exemplarily a first optical fiber 4011, a second optical fiber 4012, and a third optical fiber 4013. It should be understood that the number of optical fibers can be more than or less than three, as exemplified herein.

For example, the optical circulator 403 may be optically coupled to the coherent light source 102 and the detector 106, and further to the optical switch 601, which may correspond to the optical switch 501. In other words, the optical switch 601 may be a single-pole-multiple-throw switch, where the optical circulator 403 may be coupled to the pole port of the optical switch 601 and the first optical fiber 4011, the second optical fiber 4012, and the third optical fiber 4013 may be respectively coupled to the throw ports of the optical switch 601.

In this regard, the optical circulator 403 may receive the coherent light signal 103 from the coherent light source 102, and may transmit the coherent light signal 103 to the first optical fiber 4011, the second optical fiber 4012, and the third optical fiber 4013 via the optical switch 601, especially in a time-multiplexed manner. For example, the coherent light signal 103 may correspond to the coherent light signal 103 of the systems 100, 300, and 400.

For instance, during a first timing operation of the optical switch 601, the first optical fiber 4011 may receive and may transmit the coherent light signal 103 through the length of the first optical fiber 4011. The first optical fiber 4011 may be further in contact with a first liquid sample 1011, e.g., an end of the first optical fiber 4011 opposite to the end receiving the coherent light signal 103 may be optically coupled with or be immersed in the first liquid sample 1011.

Accordingly, the first optical fiber 4011 may transmit the coherent light signal 103 to the first liquid sample 1011 and may receive the return light signal 105, e.g., a back reflected and/or scattered light signal, from the first liquid sample 1011 in response to the coherent light signal 103. The back reflected and/or scattered lights may be in the same spatial mode of the coherent light signal 103.

Similarly, during a second timing operation of the optical switch 601, the second optical fiber 4012 may receive and may transmit the coherent light signal 103 through the length of the second optical fiber 4012. The second optical fiber 4012 may be further in contact with a second liquid sample 1012, e.g., an end of the second optical fiber 4012 opposite to the end receiving the coherent light signal 103 may be optically coupled with or be immersed in the second liquid sample 1012.

Accordingly, the second optical fiber 4012 may transmit the coherent light signal 103 to the second liquid sample 1012 and may receive the return light signal 105, e.g., a back reflected and/or scattered light signal, from the second liquid sample 1012 in response to the coherent light signal 103. The back reflected and/or scattered lights may be in the same spatial mode of the coherent light signal 103.

Similarly, during a third timing operation of the optical switch 601, the third optical fiber 4013 may receive and may transmit the coherent light signal 103 through the length of the third optical fiber 4013. The third optical fiber 4013 may be further in contact with a third liquid sample 1013, e.g., an end of the third optical fiber 4013 opposite to the end receiving the coherent light signal 103 may be optically coupled with or be immersed in the third liquid sample 1013.

Accordingly, the third optical fiber 4013 may transmit the coherent light signal 103 to the third liquid sample 1013 and may receive the return light signal 105, e.g., a back reflected and/or scattered light signal, from the third liquid sample 1013 in response to the coherent light signal 103. The back reflected and/or scattered lights may be in the same spatial mode of the coherent light signal 103.

For example, the optical circulator 403 may receive the return light signal 105 from the first optical fiber 4011, the second optical fiber 4012, and the third optical fiber 4013 via the optical switch 501, and may accordingly transmit the return light signal 105 to the detector 106, especially in the time-multiplexed manner.

For example, the detector 106 may simultaneously measure refractive index and turbidity of the first liquid sample 1011 from the return light signal 105 during the first timing operation of the optical switch 601, may simultaneously measure refractive index and turbidity of the second liquid sample 1012 from the return light signal 105 during the second timing operation of the optical switch 601, and may simultaneously measure refractive index and turbidity of the third liquid sample 1013 from the return light signal 105 during the third timing operation of the optical switch 601.

Although not shown, the system 500 may additionally comprise the signal generator 301 of FIG. 3 in order to modulate the coherent light source 102. In this regard, the coherent light signal 103 may be modulated at different frequencies.

Although not shown, each the first optical fiber 4011, the second optical fiber 4012, and the third optical fiber 4013 may be in contact with a common liquid sample 101, e.g., as shown in FIG. 1 for the system 100. In this regard, the end of the first optical fiber 4011, the end of the second optical fiber 4012, and the end of the third optical fiber 4013 may be immersed in the liquid sample 101 at different locations, e.g., spatial surface locations.

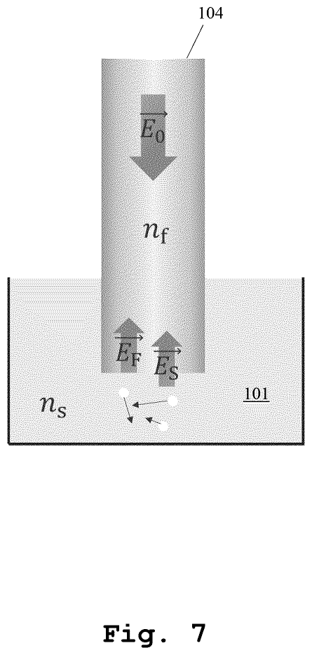

In FIG. 7, an exemplary electric field distribution in the liquid sample 101 due to reflection and scattering is illustrated. As shown in FIG. 7, the optical structure 104, especially an end of the optical structure 104, may be immersed in the liquid sample 101. In this example, n_{f} denotes the refractive index of the optical structure 104 and ns denotes the refractive index of the liquid sample 101.

Moreover, an electric field E₀ may denote the electric field of the coherent light signal 103, which may travel along the optical structure 104 with the refractive index n_{f}, and may be incident on the surface of the liquid sample 101 with the refractive index nₛ. In this regard, most of the incident light may enter into the liquid sample 101, however due to the mismatch of the refractive index of the liquid sample 101 and of the optical structure 104, a small portion may reflect at the interface.

For example, an electric field E_{F} may denote the electric field of said reflection. In addition, further differences in refractive indices coming from structures in the liquid may cause further reflections where part of the light from these further reflections may cross back over the interface. For example, an electric field of said further reflections or scattering may be denoted by an electric field Eₛ.

The detector 106 may measure the light directly reflected from the liquid sample 101 due to the illumination of the liquid sample 101 by the coherent light signal 103. The term “directly reflected light” should be understood as the light that may be reflected in the same spatial mode as the incident coherent light signal 103, i.e., 180 degrees to the incident coherent light signal 103.

For example, due to the illumination of the liquid sample 101, a Fresnel reflection with the electric field E_{F} may occur at the interface, e.g., due to the difference between the refractive indices of the liquid sample 101 and the optical structure 104. Furthermore, the amount of scattering of the light that may enter the liquid sample 101 may be dependent on the turbidity, which can be understood as the scattering of the light depending on the undissolved solids or colloids or suspended bubbles or unmixed liquid region. A part of this scattering of the light with the electric field Es may be directed in the same direction as the Fresnel reflection.

Accordingly, the resultant overall reflection ER can be written as:

$$\vec{{E}_{R}} = \vec{{E}_{F}} + \vec{{E}_{S}} \qquad ( 1 )$$

As such, the light due to the Fresnel reflection and the light from scattering may interfere, especially due to the coherence length of the light, e.g., approximately the same size as the distance between the interface and the scattering particles, or greater. Accordingly, due to the constant motion of the scattering particles in the liquid sample 101, the detector 106 may measure the overall reflection as a constant with fluctuations around said value due to the scattering.

For example, the electric fields can be expressed as sinusoidal plane waves as a function of time t in one spatial dimension z in the following manner:

$${E}_{R} = {E}_{F} + {E}_{S} = {A}_{F} cos ( k z - ω t ) + {A}_{S} cos ( k z - ω ... t + φ ) \qquad ( 2 )$$

where k is the wavenumber, ω is the angular velocity, AF is the amplitude of the Fresnel reflected light, AS is the amplitude of the scattered light, and Φ is a phase shift.

Due to the sensitivity of the photodetectors, such as the photodetector 201, to the light power rather than the electric field, the reflected power PR can be written in the following manner:

$${P}_{R} ∝ {E}_{R}^{2} = {A}_{F}^{2} {cos}^{2} ( - ω t ) + {A}_{S}^{2} {cos}^{2} ( - ω t + φ ) + 2 {A}_{F} {A}_{S} cos ( - ω t ) cos ( - ω t + φ ) \qquad ( 3 )$$

where the position of the photodetector is defined to be at z=0.

Using a suitable trigonometric identity, equation (3) can be written in the following manner:

$${P}_{R} ∝ {E}_{R}^{2} = {A}_{F}^{2} {cos}^{2} ( - ω t ) + {A}_{S}^{2} {cos}^{2} ( - ω t + φ ) + {A}_{F} {A}_{S} ( cos ( φ ) + cos ( - 2 ω t + φ ) ) \qquad ( 4 )$$

Furthermore, the terms that oscillate with an angular frequency of ω or 2ω can be replaced with their time average values, e.g., due to the fast oscillation that may not be detectable by the photodetector, in the following manner:

$${P}_{R} ∝ {E}_{R}^{2} = \frac{{A}_{F}^{2} + {A}_{S}^{2}}{2} + {A}_{F} {A}_{S} cos ( φ ) \qquad ( 5 )$$

In case the contribution from scattering is too small, i.e., A_{F}>>A_{S}, equation (5) can be reduced in the following manner:

$${P}_{R} ∝ \frac{{A}_{F}^{2}}{2} + {A}_{F} {A}_{S} cos ( φ ) \qquad ( 6 )$$

As it can be seen from equation (6), the first term is constant and may depend only on the Fresnel reflection. In the second term, cos (q) can take any value between −1 and 1, and is symmetrical around 0. The phase shift q is dependent on the position of the surfaces of the scattering particles, and may cause the fluctuations due to the constant movement of the scattering particles. In addition, the amplitude AS may be dependent on the turbidity, which may increase as the turbidity increases. Furthermore, the measurement of the Fresnel reflection can give a measurement of the refractive index of the liquid sample 101.

Therefore, from equation (6), it is possible to measure the contribution from the Fresnel reflection independently of the turbidity. For example, the simple average of the measured signal may result in the contribution only from the Fresnel reflection, and hence may give a measure of the refractive index. On the other hand, a quantification, e.g., high-pass filtration followed by a root-mean-square operation, or a suitable autocorrelation function, of the fluctuations may only correlate with the turbidity.

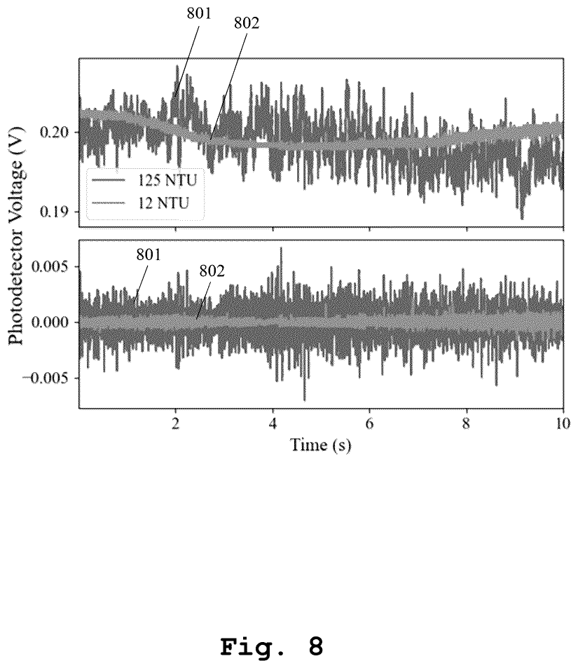

In FIG. 8, exemplary photodetector signals from two Formazin solutions 801, 802 are illustrated. The horizontal axis denotes the time in seconds and the vertical axis denotes the photodetector voltage in volts. The turbidity value for the first Formazin solution 801 is measured as 125 NTU and the turbidity value for the second Formazin solution 802 is measured as 12 NTU. The top plot corresponds to the raw photodetector signals from the Formazin solutions. The bottom plot corresponds to the same signals as the top plot, however, after applying a high-pass filter.

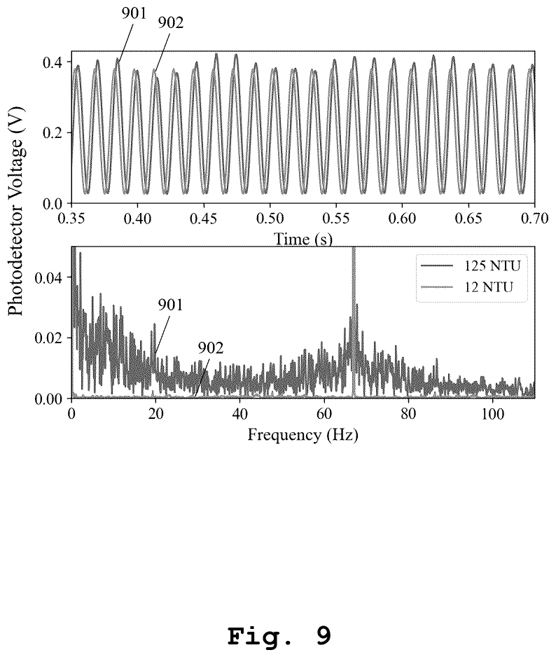

In FIG. 9, exemplary photodetector signals from two Formazin solutions 901, 902 for modulated light sources are illustrated. Particularly for a modulated light source, the signal level corresponding to the Fresnel reflection can be retrieved through a Fourier transform. In this regard, the value at the modulation frequency may be related to the Fresnel reflection, whereas the power at other frequencies may be related to the amount of turbidity.

Turning back to FIG. 9, the horizontal axis of the top plot denotes the time in seconds and the vertical axis of the top plot denotes the photodetector voltage in volts. Additionally, the horizontal axis of the bottom plot denotes the frequency in Hz and the vertical axis of the bottom plot denotes the photodetector voltage in volts. In this regard, the light source, i.e., laser, is modulated at a frequency of 67 Hz.

The top plot corresponds to the raw photodetector signals from the Formazin solutions 901, 902, which are identical to the Formazin solutions 801, 802 of FIG. 8. The bottom plot corresponds to the same signals as the top plot, however after applying a Fourier transform.

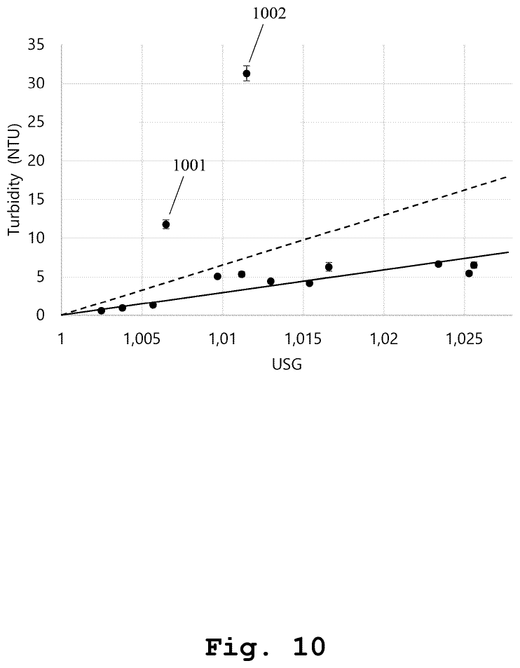

In FIG. 10, an exemplary graph of turbidity vs Urine specific gravity (USG) for human urine samples is illustrated.

For example, the liquid sample 101 may be a urine sample, and the concentration of dissolved solids in the liquid sample is a concentration of dissolved metabolites in the urine sample. In addition, the concentration of undissolved solids in the liquid sample is the concentration of secreted proteins in the urine sample.

An increased level of protein in urine (Proteinuria) may be a sign of disease. This may be due to an increase in the protein secreted into the urine, which would normally be filtered by the kidneys. However, a simple measurement of the protein level, e.g., with a spot check, may give false negatives of Proteinuria if the urine is diluted. Therefore, a clinical diagnosis may be required by taking urine over a 24-hour period and by measuring the average protein concentration.

However, a spot check would be of more value for preventative healthcare purposes, especially since in early stages, it might be possible to detect an increase in protein without symptoms. In principle, this spot check may need to measure the protein concentration while factoring out the effect of urine dilution, i.e., a combined measurement of protein and urine specific gravity (USG).

Therefore, in the case of the urine sample, the detector 106 may measure the urine turbidity to estimate the undissolved portion in the urine sample, thereby measuring the protein content and may further measure the refractive index to estimate the dissolved metabolites in the urine sample, thereby measuring the USG.

In particular, the detector 106 may perform the turbidity and refractive index measurement simultaneously, especially to simultaneously measure the secreted protein and USG, and may use their ratio as a possible parameter to yield proteinuria diagnosis.

Turning back to FIG. 10, the horizontal axis denotes USG and the vertical axis denotes turbidity in nephelometric turbidity unit (NTU). The solid line represents the normal turbidity level for a given USG and the dashed line represents the level at which proteinuria can be declared. It is to be noted that the lines can also be formed as curves or other complex functions. It can be seen that two samples 1001, 1002 are abnormal and result in a much higher turbidity to USG ratio.

Alternatively, for example, the liquid sample may be a bioreactor sample and the concentration of dissolved solids in the liquid sample is a concentration of a substrate in the bioreactor sample. Additionally, the concentration of undissolved solids in the liquid sample is a concentration of cells in suspension in the bioreactor sample.

For example, in optimal conditions, the rate of cell growth in a bioreactor can be expressed by the Monod equation for cell growth in the following manner:

$$r = {μ}_{max} \frac{xS}{K + S} \qquad ( 7 )$$

where r is the volumetric rate of cell growth, μₘₐₓ is the maximum specific growth rate, K is the saturation constant, x is the cell concentration and S is the substrate concentration, e.g., the concentration of nutrient or food for cells.

In this regard, during the process, the variables are x and S, while the other terms are constants. Therefore, measurements of x and S may result in a calculation of the optimal growth rate. If the measured actual growth rate, i.e., the change in x over time, is significantly lower than the calculated optimal growth rate, this could act as an early warning for process control.

Furthermore, the cells in suspension may increase the turbidity of the liquid, which may result in the measurement of cell density. In addition, the cell growth substrate may consist of dissolved solids, such as sugars, which may alter the refractive index. This may result in the measurement of the substrate concentration.

Therefore, in the case of the bioreactor sample, the detector 106 may measure the turbidity to estimate the undissolved solids, i.e., the cells in suspension, in the bioreactor sample, thereby measuring the cell density and may further measure the refractive index to estimate the dissolved solids, e.g., sugars, in the bioreactor sample, thereby measuring the substrate concentration. Accordingly, the optimal growth rate can be calculated with simultaneous measurements of turbidity and refractive index.

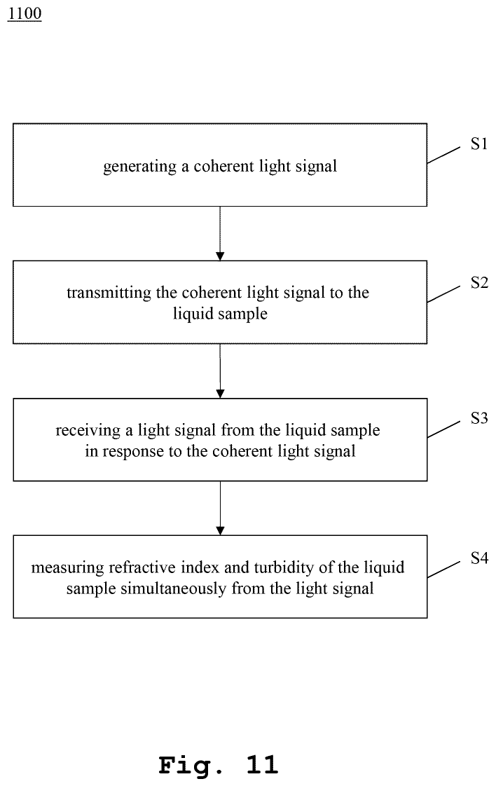

In FIG. 11, an exemplary embodiment of the method 1100 according to the present disclosure. In a first step S1, a coherent light signal is generated. In a second step S2, the coherent light signal is transmitted to the liquid sample. In a third step S3, a light signal is received from the liquid sample in response to the coherent light signal. In a fourth step S4, refractive index and turbidity of the liquid sample are simultaneously measured from the light signal.

Therefore, the present disclosure facilitates the measurement or estimation of undissolved solids in reference to the dissolved solids or vice versa in a liquid sample, especially through the measurement of back reflected and/or scattered coherent light. The amount of fluctuation in the measured signal may correlate with turbidity, while the average signal level may correlate with the Fresnel reflection, which may result in simultaneous measurements of turbidity and refractive index. It is to be noted that the measurement of refractive index can be understood as the measurement of refractive index of the liquid sample between the particles that cause turbidity.

Furthermore, turbidity may be caused by the presence of the undissolved solids while the refractive index may be changed by the dissolved solids, which may allow for the simultaneous measurement or estimation of the quantity of these two types of solids in the liquid sample.

It is important to note that, in the description as well as in the claims, the word “comprising” does not exclude other elements or steps and the indefinite article “a” or “an” does not exclude a plurality. A single element or other unit may fulfill the functions of several entities or items recited in the claims. It should be understood that the term “and/or” used in the specification and the appended claims of this application refers to any combination and all possible combinations of one or more associated listed items, and includes these combinations.

It should also be understood that the word “coupled” implies that the elements may be directly connected together or may be coupled through one or more intervening elements. Moreover, the disclosure with regard to any of the aspects is also relevant with regard to the other aspects of the disclosure.

Although the disclosure has been illustrated and described with respect to one or more implementations, equivalent alterations and modifications will occur to others skilled in the art upon the reading and understanding of this specification and the annexed drawings. In addition, while a particular feature of the disclosure may have been disclosed with respect to only one of several implementations, such feature may be combined with one or more other features of the other implementations as may be desired and advantageous for any given or particular application.

What is claimed is:

1. A system for simultaneously measuring refractive index and turbidity of a liquid sample, the system comprises:
a coherent light source configured to generate a coherent light signal,
an optical structure being in contact with the liquid sample, the optical structure is configured to transmit the coherent light signal to the liquid sample and further to receive a light signal from the liquid sample in response to the coherent light signal, and
a detector configured to receive the light signal and further to measure refractive index and turbidity of the liquid sample simultaneously from the light signal.

2. The system according to claim 1,
wherein the detector is further configured to measure or estimate a concentration of dissolved solids in the liquid sample from the refractive index measurement, and/or
wherein the detector is further configured to measure or estimate a concentration of undissolved solids in the liquid sample from the turbidity measurement.

3. The system according to claim 2,
wherein the concentration of dissolved solids in the liquid sample is a concentration of dissolved metabolites in a urine sample, and
wherein the concentration of undissolved solids in the liquid sample is a concentration of secreted proteins in the urine sample.

4. The system according to claim 2,
wherein the concentration of dissolved solids in the liquid sample is a concentration of a substrate in a bioreactor, and
wherein the concentration of undissolved solids in the liquid sample is a concentration of cells in suspension in the bioreactor.

5. The system according to claim 4, wherein the light signal comprises one or more lights reflected at a surface of the liquid sample and/or one or more lights scattered within the liquid sample, especially in response to the coherent light signal.

6. The system according to claim 5, wherein the optical structure comprises a single optical fiber configured to transmit the coherent light signal to the liquid sample and further to receive the light signal from the liquid sample.

7. The system according to claim 6, wherein the single optical fiber is configured such that an end of the single optical fiber is immersed in the liquid sample.

8. The system according to claim 6, wherein the single optical fiber is a single mode optical fiber.

9. The system according to claim 8, wherein the single optical fiber is configured such that an end of the single optical fiber is immersed in the liquid sample.

10. The system according to claim 9, wherein the optical structure comprises an optical circulator configured to direct the coherent light signal towards the liquid sample and further to direct the light signal from the liquid sample.

11. The system according to claim 10, wherein the system further comprises a signal generator configured to modulate the coherent light source at a predefined frequency.

12. The system according to claim 1, wherein the system comprises:
an optical switch, and
a plurality of optical structures,
wherein the optical switch is configured such that each of the plurality of optical structures transmits a respective coherent light signal from the coherent light source to the liquid sample and further to receive a respective light signal from the liquid sample in response to the coherent light signal, and
wherein the detector is configured to receive the light signals from the plurality of optical structures and further to measure refractive index and turbidity of the liquid sample simultaneously from each of the light signals.

13. The system according to claim 1, wherein the detector is configured to measure a resultant reflection from the light signal corresponding to an interference between lights due to Fresnel reflection and lights from scattering in the liquid sample, especially due to the transmission of the coherent light signal to the liquid sample.

14. The system according to claim 13,
wherein the detector is configured to measure the Fresnel reflection by averaging the measured resultant reflection in order to measure the refractive index of the liquid sample, and/or
wherein the detector is configured to measure the scattering by filtering the measured resultant reflection in order to measure the turbidity of the liquid sample.

15. The system according to claim 1, wherein the light signal comprises one or more lights reflected at a surface of the liquid sample and/or one or more lights scattered within the liquid sample, especially in response to the coherent light signal.

16. The system according to claim 1, wherein the optical structure comprises a single optical fiber configured to transmit the coherent light signal to the liquid sample and further to receive the light signal from the liquid sample.

17. The system according to claim 1, wherein the optical structure comprises an optical circulator configured to direct the coherent light signal towards the liquid sample and further to direct the light signal from the liquid sample.

18. The system according to claim 1, wherein the system further comprises a signal generator configured to modulate the coherent light source at a predefined frequency.

19. A method for simultaneously measuring refractive index and turbidity of a liquid sample, the method comprises:
generating a coherent light signal,
transmitting the coherent light signal to the liquid sample,
receiving a light signal from the liquid sample in response to the coherent light signal, and
measuring refractive index and turbidity of the liquid sample simultaneously from the light signal.

20. The method according to claim 19, wherein the method further comprises:
measuring or estimating a concentration of dissolved solids in the liquid sample from the refractive index measurement, and/or
measuring or estimating a concentration of undissolved solids in the liquid sample from the turbidity measurement.
